(19) United States
(12) Patent Application Publication
(10) Pub. No.: US 2023/0026966 A1
(43) Pub. Date: Jan. 26, 2023

(54) MACHINE, SYSTEM AND METHOD FOR THE FILTRATION, DISINFECTION AND PURIFICATION OF FUEL IN CONTAMINATED TANKS

(71) Applicant: FDR PATENT, S.L., Granada (ES)

(72) Inventors: Ana Maria ACIEN REYES, Granada (ES); Sergio Julio MORILLAS VALERO, Granada (ES)

(21) Appl. No.: 17/778,697

(22) PCT Filed: May 31, 2021
(86) PCT No.: PCT/ES2021/070388

§ 371 Date: May 20, 2022

(30) Foreign Application Priority Data
Jun. 3, 2020 (ES) U202031130
May 19, 2021 (ES) P202130452

(51) Int. Cl. F02M 37/34; B01D 35/26; B01D 17/02; B01D 35/143; B01D 35/027; B01D 37/00; B08B 9/093; F02M 37/24; F02M 27/06; F02B 77/04; A61L 2/26; A61L 2/02; A61L 2/10

(52) U.S. Cl. CPC F02M 37/34; B01D 35/26; B01D 17/0217; B01D 35/143; B01D 35/027; B01D 37/00; B08B 9/0936; F02M 37/24; F02M 27/06; F02B 77/04; A61L 2/26; A61L 2/022; A61L 2/10; B08B 2209/08; F02B 2077/06

(57) ABSTRACT

A machine, a filtration system and a method for disinfection and purification in fuel tanks located in places with high humidity or high relative humidity, having a machine (1) with an inlet for the fuel from the tank, a filtration system of a coarse-grain filter (2) and a fine-grain filter (2′); a radiation chamber (3) configurable for the application of radiation to the filtered fuel; a pump (4); a flowmeter (5) with at least two electrovalves (17); a centrifuge (6) with a turbine filter (6′) and control via an electrical panel (7); a cleaning machine (1); a suction hose (8) with an extendable suction lance (9); a high-pressure discharge hose (11); a return hose (12); a system for the opening and sealing of the tank (25); and an automatic filter self-cleaning system which reverses the flow, sending the fouled fuel to an additional tank (14) where it is drained.

### OBJECT OF THE INVENTION

The present invention discloses a machine, system and method for the disinfection and purification in fuel tanks located in places with high humidity or high relative humidity, preventing the proliferation of bacteria and fungi which, in addition to contaminating fuels, particularly affects the metals used to manufacture the tank.

### BACKGROUND OF THE INVENTION

At present, in any fuel tank located in an environment with high relative humidity, when the fuel flows out, moist air inevitably enters the tank, creating over time due to condensation of said humidity a layer of water that is deposited at the bottom of the tank and can reach the lowest nozzle where the fuel flows out.

It is precisely on the contact surface between the water that has been deposited at the bottom of the fuel tank and the fuel itself where the bacteria that give rise to algae are produced and end up contaminating all the fuel and rendering the systems where the fuel itself is used (filters, engines, etc.) unusable. Algae are highly corrosive and grow in the tank, even perforating the fuel tank and clogging engine filters.

In the field of the naval industry, where high relative humidity accentuates this problem, only chemical products are used to solve the problem of algae due to biological contamination in fuel tanks. However, these chemicals are not effective, and manual cleaning and disinfection is very expensive.

Fuels stored for long periods of time can undergo polymerization, oxidation, emulsions, and bacterial and fungal contamination. These problems can form residues. The aging of these fuel residues and fuel lines can cause serious problems in engines and fuel systems. Bacteria and fungi can grow and develop in crude oil and fuel storage tanks. Biologically contaminated storage tanks experience problems related to quality and infrastructure. Biologically contaminated storage tanks experience problems related to fuel quality and fuel storage infrastructure and engine operating system. Microbial contamination problems cause the proliferation of algae or biological mass in a gelatinous form, which is highly corrosive, clogging engine filters, pipes and injectors and literally devouring the metals in the tank that contains it, in addition to reducing the quality of the fuel itself.

Today, in the face of such fuel contamination, the solution that guarantees the best solution to the problem involves discarding the contaminated fuel and manually cleaning the tank, an extremely costly process, since the problem will return in a short period of time because the tank normally cannot be cleaned 100%. In addition to this, in the case of fuel tanks located on ships and pleasure craft, the tanks are not simple cylindrical or four-walled chambers, but are integrated into the structure of the ship, such as keels, bulkheads, stringers, cross frames and other reinforcing members, which extend into the spaces of the tank, therefore cleaning them is extremely important since the integrity of the ship or pleasure craft depends on the health of the fuel tank.

Fuel tanks must always be clean of contamination so that the fuel has an acceptable quality, therefore, in the event that there is a contaminated tank, it is necessary to disinfect it in order to guarantee this objective.

In the state of the art, patent application WO/2004/103586 discloses a device for interior flushing of spaces in containers, e.g. ship's tanks, wherein at least one flushing-liquid pipe for the supply of flushing liquid is provided with at least one flushing nozzle for discharging jets of flushing liquid, and wherein a driving or operating device is provided to rotate the flushing-liquid pipe about a geometric rotation axis and the flushing nozzle about another geometric rotation axis, the driving device being provided to be brought to rotate in opposite rotary directions by reversing its rotary direction. A switch device is provided to bring either the flushing-liquid pipe or the flushing nozzle to rotate in dependence of the rotary direction of the driving device, and a control device is provided to allow control of the driving device to rotate in one or the opposite rotary direction.

European patent EP0723909 relates to a flushing device for internal flushing of tanks, preferably tanks in vessels or ships, whereby at least one flush-liquid pipe is rotatably provided in the tank, whereby the flush-liquid pipe has at least one flush nozzle which is pivotally mounted relative to the flush-liquid pipe, whereby a supply conduit is provided to feed flush liquid to the flush-liquid pipe and through said pipe to the flush nozzle which is adapted to direct jets of flush liquid towards the inner sides of the tank, whereby a turbine wheel in a turbine device is mounted in the supply conduit so that said wheel is driven by the flow of flush liquid in said supply conduit and whereby the turbine wheel is provided to drive a driving assembly located outside the supply conduit and provided to bring the flush-liquid pipe to rotate and at the same time revolve or turn the flush nozzle.

Such methods are known in the most varied embodiments of the prior art. However, all of them are quite time-consuming and laborious and also costly because different problems arise. Systems today for the cleaning and disinfection of fuel tanks are based on biocidal agents, which are inefficient and costly for the results obtained. Over the years and due to the humidity of the environment, fuel storage tanks accumulate a layer of water at their base that causes the development and proliferation of a series of microorganisms, giving rise to a dense and filamentous algae that, at best it clogs the engine filters and stops the engine. These microorganisms are corrosive and damage the tank that contains them, even puncturing it, which is critical on a craft. The effects of dirt in fuels can damage the engine and other important parts like injectors.

The present invention solves the problem in a satisfactory manner, since it incorporates a machine and an integral system, which comprises the filters necessary and sized for the tank and the degree of contamination, the objective of which is for the fuel to reach the radiation chamber clean, which will be configurable for the application on the fuel of the necessary radiation by means of different light spectra, including full spectrum. Taking into account that the system must be adapted to a multitude of types of tanks and contamination levels, in the most demanding systems, it must also incorporate a pump responsible for circulating the fuel inside, a flowmeter, a centrifuge that incorporates a turbine filter to remove the water contained in the fuel, an electrical panel where all the functions of the device are controlled; a suction hose connected to an extendable suction lance that is finished off at its end by a rotating jet; a high-pressure discharge hose; system for the opening and sealing of a tank; an automatic filter self-cleaning system by reversing the fluid and an absorption tube with a grinding propeller.

The system ensures comprehensive cleaning and disinfection of fuel located in places with medium or high relative humidity, and offers the possibility of removing existing chemicals by means of variable radiation.

### DESCRIPTION OF THE INVENTION

A first aspect of the invention is based on a machine for a system for the cleaning and disinfection for fuel tanks. The invention is applicable to any fuel tank susceptible to biological contamination (in fuel recycling plants, workshops, etc.), although its application is of particular relevance in large ships and all types of pleasure craft, as the proper operation of the engine is of vital importance in these cases. The machine comprises a filtration system configured to filter the fuel received through the intake port, a radiation chamber configured to apply ultraviolet or infrared light on the filtered fuel, a pump responsible for circulating the fuel inside the machine (preferably a self-priming centrifugal pump), and a high-pressure fuel outlet port. In turn, the filtration system preferably comprises a coarse-particle filter, a fine-particle filter and a fluid separator filter. In one embodiment, the coarse-particle filter is configured to filter solid impurities of a size larger than 20 μm, and the fine-particle filter is configured to filter solid impurities of a size less than 20 μm. The fluid separating filter is preferably a water separating filter.

In another aspect of the invention, a complete cleaning system is disclosed, which incorporates the machine disclosed in the first aspect of the invention, a high-pressure discharge hose that is finished off at its end by a rotating jet; a system for the opening and sealing of the tank; an automatic filter self-cleaning system by reversing the fluid and a radiation chamber configured for ultraviolet A, B, C and/or infrared light. The system ensures comprehensive cleaning and disinfection of fuel located in places with medium or high relative humidity, and offers the possibility of removing microorganisms by means of variable radiation.

In another aspect of the invention, a method for the filtration, disinfection and recycling of fuel in contaminated tanks is shown, which incorporates several operating stages such as the suctioning of the contents of the tank, by means of the suction hose; the separation of fuels with the different filters, the removal of water present in the fuel, radiation with ultraviolet and/or infrared rays and reconversion of the fuel to the tank by means of a high-pressure hose with enough pressure to cause a rotating high-pressure jet installed at the end of said hose to work.

### DESCRIPTION OF THE DRAWINGS

To complement the description provided herein and for the purpose of helping to better understand the features of the invention according to a preferred practical embodiment thereof, said description is accompanied by a set of figures constituting an integral part of the same, wherein the following is depicted with an illustrative and non-limiting character:

FIG. 1 shows an external view of the fuel tank with the machine (1) coupled.

FIG. 2 shows an inside view of the tank, where the machine (1) is shown coupled with the suction hose (8) and the extendable lance (9).

FIG. 2.1 shows a detailed view of the return hose with a rotating jet (10).

FIG. 2.2 shows a detailed view of the absorption tube with a grinding propeller (20).

FIG. 3 shows an inside view of the basic elements of the machine (1), where the elements are shown, such as the filtration system comprised of a coarse-grain filter (2) and a fine-grain filter (2′), the radiation chamber (3), the pump (4) responsible for circulating the fuel inside, the flowmeter (5), the centrifuge (6) that incorporates a turbine filter (6′) to remove the water contained in the fuel and the electrical panel (7).

FIG. 4 shows a detail of the radiation chamber (3) comprised of panels (18) of LEDs (23) with different wavelengths and a magnifying lens (24), a transparent tank with a fuel circulation circuit (19), cooling fans (21) and a box containing the components (22).

FIG. 4.1 shows a detail of the cooling fan (21).

FIG. 4.2 shows a detail of a LED (23) with a magnifying lens (24).

FIG. 4.3 shows a detail of the box containing the radiation chamber (22).

FIG. 5 shows a detailed view of the system for the opening and sealing of the tank (13) and the hermetic closure system (25) at access points to the fuel tank.

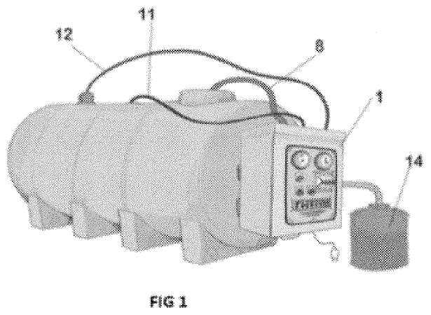

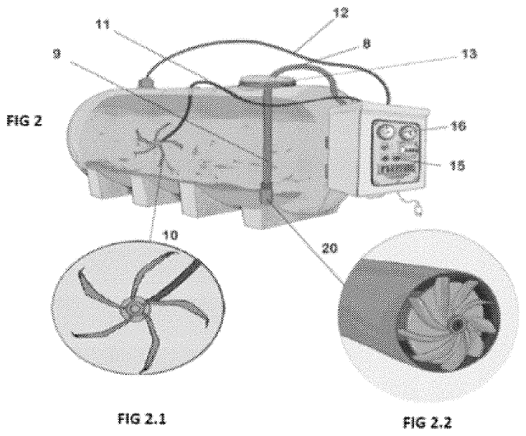

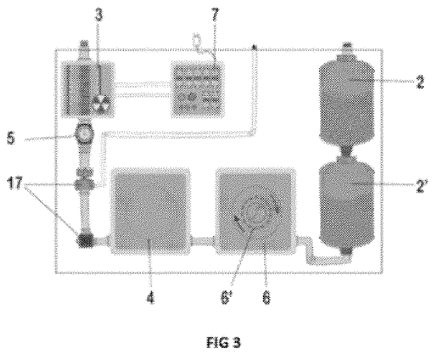

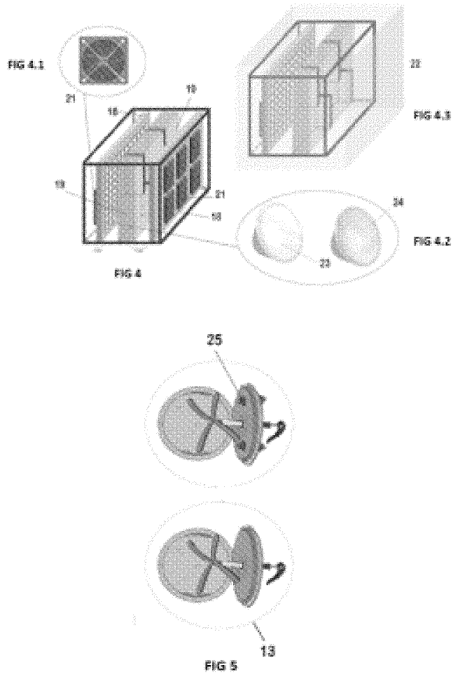

### PREFERRED EMBODIMENT OF THE INVENTION

The present embodiment discloses a machine that forms an integral part of a system for cleaning and disinfection in fuel tanks. The invention is applicable to any fuel tank susceptible to biological contamination (in fuel recycling plants, workshops, etc.), although its application is of particular relevance in large ships and all types of pleasure craft, as the proper operation of the engine is of vital importance in these cases.

The fuel from the fuel tank is introduced inside the machine (1) through a suction hose (8), wherein said machine (1) comprises an inlet for the fuel from the tank; a filtration system comprised of a coarse-grain filter (2) and a fine-grain filter (2′) configured to filter the fuel received through the intake port; a radiation chamber (3), configurable for the application of ultraviolet light type A, B, C with different wavelength combinations, since ultraviolet light can be used successfully as a germicide and bactericide and allows microorganisms, such as bacteria, viruses and other pathogens to be killed, or prevent their development and provides a chemical-free alternative to other methods for disinfection, wherein the radiation chamber (3) can also be configured for use by means of infrared rays on the filtered fuel, with different wavelength combinations; a pump (4) responsible for circulating the fuel inside said machine (1); a flowmeter (5), with at least two electrovalves (17), which allow the flow rate passing through the radiation chamber (3) to be regulated; a centrifuge (6) with a turbine filter (6′) to remove the water contained in the fuel, as well as any particle that may have surpassed the filtration system; and control means via an electrical panel (7) that is used to take measurements of the temperature in the radiation chamber (3), flow rates, radiated litres, etc., as well as to activate or deactivate each of the components of the system.

The radiation chamber (3) is designed to work with high radiation power and specifically for fuel, and given its resistance it can be used with other fluids. In addition, it allows the wavelength to be regulated, being able to simultaneously use the desired frequencies, even radiating with the full spectrum. With this functionality it is possible to remove specific parasites, to remove all biological contamination. The radiation chamber is comprised of panels (18) with a plurality of LEDs (23) with different wavelengths and a magnifying lens (24), a transparent tank with a fuel circulation circuit (19), cooling fans (21) and a box containing the components (22). By using LED technology, the low-consumption radiation chamber (3) can work in addition to 220 v at 12 or 24 v in ships, vehicles or machinery, without detriment to performance. It is also achieved that the system generates little heat, since, being a matrix of radiation sources, if it is done with conventional technology, the temperature that would be generated would become a serious problem.

The machine (1) also incorporates a variable flow control system by means of electrovalves, which allow the fuel to pass more slowly or more quickly through the radiation chamber, this allows the fuel to be controlled, since depending on the purpose desired, the radiation time is very important; it additionally incorporates an adjustable ventilation system (15) with temperature gauges (16) that allows the temperature to be controlled in the desired range throughout the duration of the process.

In another aspect of the invention, the machine (1) forms part of an integral system the filtration, disinfection and purification of fuel that aims to solve a problem that exists in all fuel tanks located in places with a medium or high relative humidity. This humidity causes an unwanted amount of water to accumulate in tanks by condensation at the bottom, which over time will develop a proliferation of bacteria and fungi that are corrosive, and that in the case of tanks that have an engine, cause problems of obstruction in the fuel absorption system, filters, pipes and injectors, triggering the stoppage of the engine and problems of metal corrosion, this problem being especially serious in ships, since the fuel tank is part of the hull itself, and corrosion can cause the ship to sink.

The system for the filtration, disinfection and purification of fuel in contaminated tanks comprises a cleaning machine (1) that incorporates a fuel intake port; a suction hose (8) for directing the suctioned fuel towards the fuel intake port and connected to an extendable suction lance (9), configured so that once inserted inside the fuel tank it adapts its length, allowing fuel to be suctioned from the bottom of said fuel tank and finished off at its end with a grinding propeller (20) that prevents residues from clogging the system; a high-pressure discharge hose (11) to direct fuel at high pressure from the fuel outlet of the machine (1) to the fuel tank and is connected at its end located inside the tank to a rotating jet which rotates and cleans the walls and bottom of the tank as the fuel under pressure flows out; a return hose (12), which removes excess pressure by returning excess fuel to the tank; a system for the opening and sealing of the tank (25) with connectors for the suction hose (8) and return hose (12), which prevents odours and spills while working on cleaning and disinfection; a system for the opening and sealing of the final tank (13) with an hermetic closure and easy opening so that the tank is sealed and practicable on future occasions. In addition, the system allows the incorporation of an automatic filter self-cleaning system which reverses the flow, sending the fouled fuel to an additional tank (14) where the amount of fuel that has been used to clean the filters is drained and becomes usable again.

The system of the invention also optionally has sensors/indicators for changing or cleaning filters, for the temperature and for the process time. Fuse box and magneto-thermal circuit breakers of adequate power for the model. Panel of switches and control instruments, in the case of fixed models it has a panel that is installed in the ship or vehicle, in transportable models it is incorporated in the machine itself (1).

In another aspect of the invention, a method for the filtration, disinfection and recycling of fuel in contaminated tanks is disclosed, which incorporates several operating stages such as the suctioning of the contents of the tank, by means of the suction hose; the separation of fuels with the different filters, the removal of water present in the fuel, radiation with ultraviolet and/or infrared rays and reconversion of the fuel to the tank by means of a high-pressure hose with enough pressure to cause a rotating high-pressure jet installed at the end of said hose to work.

The method begins with a first stage that is the suctioning of the liquid contained within the fuel tank from the deepest part thereof, with the suction hose (8) and by applying an extendable suction lance (9) configured to, once inserted inside a fuel tank, suction fuel from the bottom of said tank. At this stage, depending on the requirements of the version of the machine that is needed, it can incorporate a pneumatic or electric grinding propeller (20) at the absorption end, as well as a hermetic closing system (25) at access points to the fuel tank that consists of an cover that can be adjusted to the hole made with a lower claw and rubber bushings for a perfect closure. Said cover will have internal quick connections for all the devices that must be inserted in the tank and external quick connections for the connection with the machine. Likewise, at this stage, depending on the machine model being built, a self-priming pump of between 12 v and 220 v and with adequate power for recirculation may be implemented, in the case of machines that are permanently installed next to the tank, or with high absorption capacity if they must extract the fuel from a certain height.

Once the contents of the tank have been suctioned up by the machine (1), it passes to a second stage where the fuel is filtered, separating biological residues and coarse solid impurities of a size larger than 20 microns from the fuel.

Once the fuel has been filtered, it goes to a third stage where a new fuel filtering is carried out, this time with fine filters of a size of less than 20 microns and up to 2 microns so as not to leave any solid residue. At this stage, when the system is not fixed, a filter self-cleaning system can alternatively be incorporated which consists of reversing the circulation flow, so that by means of electro-valves (17) suction continues from the absorption intake and the fuel is returned to an additional tank that separates the dirt and fuel by decantation, so that no fuel is lost in the cleaning process; when the system restores normal flow, the outlet valve continues for a few seconds emptying fuel into the cleaning tank to clean the circuit completely, and after that time it returns to the normal operating position, causing the fuel to pass to the next phase.

After filtering, a fourth stage is carried out to remove the water that is contained in the fuel by means of the centrifuge (6) with a turbine filter (6′) or only a water separating turbine. The water will be dislodged from the system through a drainage nozzle located in the lower part of the centrifuge (6) or the water separating turbine and only the fuel continues to be treated.

When the fuel is completely clean, a fifth stage is carried out which consists of irradiating the fuel with ultraviolet and/or infrared rays inside the radiation chamber (3). Depending on the objective, the user can configure the type of radiation with different wavelengths between 200 nm and 800 nm, or provide it with the full spectrum, to kill living organisms or to remove chemicals or dyes. Also in this stage and for large capacity models, the control of the flow rate in the radiation chamber (3) is included, being able to adjust the speed of passage through the chamber and consequently the time of exposure to said radiation. The surplus fuel, when the selected flow rate is small, returns to the tank after the filtration process without going through the radiation chamber (3). To optimize, it is recommended to use a different tank for the fuel (14) that has passed through the entire process and not recirculate. Given the low permeability of the fuel to radiation, the radiation chamber (3) also has micro-chambers through which the fuel circulates with the appropriate thickness for the radiation to be effective.

Finally, in a sixth stage, the fuel is returned to the tank through the high-pressure discharge hose (11) with enough pressure to cause a rotating high-pressure jet (10) installed at the end of said hose (11) to work so that it helps to clean the tank.

The materials used are adequate and suitable for handling fuels and have adequate electrical conductions to absorb the static electricity generated in the process. Both the frame and the type of pump, filters and other components are adaptable according to the machine model and its requirements, for example, the machine that is in a port to serve many ships, in addition to being able to move, must be much more powerful than the one permanently installed on a ship. On the contrary, the latter must be smaller and be able to be coupled next to the tank.

The present invention has the following advantages:

Installed in ships and pleasure craft without biological contamination in their fuel tank, it prevents said contamination and keeps it clean of impurities with 100% effectiveness.

In the case of ships and pleasure craft that, out of necessity, refuel with low-quality fuel, the use of this system allows the fuel to be refined in such a way that it does not foul the engine filters or damage the engine due to impurities.

In the case of ships and pleasure craft with biological contamination in the fuel tank or one that is very fouled, the system effectively recycles the fuel, although it will require more operating time and frequent cleaning of the filters.

It is applicable for fuel recycling plants, so that the contaminated and fouled fuel becomes usable again.

Due to its versatility, it can be used in a portable manner for machinery on construction sites, in the field, and in places where the fuel is fouled, changing the 5-outlet jet system for a flexible hose for fuel to flow out with an idle jet at the end.

The system can be implemented in workshops where residual diesel is produced.

This system is especially useful in fuel tanks for large ships and all types of craft, vehicles and machinery in coastal areas, and any type of fuel tank located in places with high relative humidity.

1. A machine for the filtration, disinfection and purification of fuel in contaminated tanks, comprising:
an inlet for the fuel from the tank;
a filtration system comprised of a coarse-grain filter (2) and a fine-grain filter (2′) configured to filter the fuel received through the intake port;
a radiation chamber (3) configured to apply radiation on the filtered fuel;
a pump (4) responsible for circulating the fuel inside said machine (1);
a flowmeter (5), with at least two electrovalves (17), which allow the flow rate passing through the radiation chamber (3) to be regulated;
a centrifuge (6) with a turbine filter (6′) to remove the water contained in the fuel; and
control means via an electrical panel (7) that is used to take measurements of the temperature in the radiation chamber (3), flow rates, radiated litres, as well as to activate or deactivate each of the components of the system.

2. The machine for the filtration, disinfection and purification of fuel in contaminated tanks according to claim 1, wherein the radiation chamber (3) allows the application of ultraviolet light type A, B, C with different wavelength combinations.

3. The machine for the filtration, disinfection and purification of fuel in contaminated tanks according to claim 1, wherein the radiation chamber (3) allows the application of infrared rays on the filtered fuel, with different wavelength combinations.

4. The machine for the filtration, disinfection and purification of fuel in contaminated tanks according to claim 1, wherein the radiation chamber (3) incorporates panels (18) with a plurality of LEDs (23) with different wavelengths and a magnifying lens (24), a transparent tank with a fuel circulation circuit (19), cooling fans (21) and a box containing the components (22).

5. The machine for the filtration, disinfection and purification of fuel in contaminated tanks according to claim 1, further comprising an adjustable ventilation system (15) with temperature gauges (16) that allow the temperature to controlled in a range desired throughout a duration of a process.

6. The machine for the filtration, disinfection and purification of fuel in contaminated tanks according to claim 1, further comprising sensors/indicators for changing or cleaning filters, for the temperature and for the process time.

7. A system for the filtration, disinfection and purification of fuel in contaminated tanks, comprising:
the cleaning machine (1) according to claim 1 incorporating a fuel intake port;
a suction hose (8) for directing the suctioned fuel towards the fuel intake port and connected to an extendable suction lance (9);
a high-pressure discharge hose (11) for directing high-pressure fuel from the fuel outlet port of the machine (1) to the fuel tank;
a return hose (12), which removes excess pressure by returning excess fuel to the tank;
a system for the opening and sealing of the tank (25) with connectors for the suction hose (8) and return hose (12), which prevents odors and spills while working on cleaning and disinfection;
a system for the opening and sealing of the final tank (13) with a hermetic closure so that the tank is sealed; and
an automatic filter self-cleaning system which reverses the flow, sending the fouled fuel to an additional tank (14) where it is drained.

8. The system for the filtration, disinfection and purification of fuel in contaminated tanks according to claim 7, wherein the extendable suction lance (9) is configured so that once inserted inside the fuel tank it adapts its length, allowing fuel to be suctioned from the bottom of said fuel tank.

9. The system for the filtration, disinfection and purification of fuel in contaminated tanks according to claim 7, wherein the extendable suction lance (9) is finished off at its end with a grinding propeller (20) that prevents residues from clogging the system.

10. The system for the filtration, disinfection and purification of fuel in contaminated tanks according to claim 7, wherein the discharge hose (11) is connected at its end located inside the tank to a rotating jet that allows the rotation and the cleaning of the walls and bottom of the tank as the fuel under pressure flows out.

11. The system for the filtration, disinfection and purification of fuel in contaminated tanks according to claim 7, wherein it has sensors/indicators for changing or cleaning filters, for the temperature and for the process time.

12. A method for the filtration, disinfection and purification of fuel in contaminated tanks using the system according to claim 7 comprising the following operating stages:
a first stage consisting of suctioning the contents of the tank, by means of the suction hose (8) and an extendable suction lance (9);
a second stage of filtering with the coarse filter (2), separating biological residues and coarse solid impurities of a size larger than 20 microns from the fuel;
a third stage of newly filtering the fuel, with the fine filter (2′) of a size of less than 20 microns and up to 2 microns of solid residue;
a fourth stage of removing the water contained in the fuel by means of the turbine filter (6′) arranged in the centrifuge (6);
a fifth stage of radiating with ultraviolet and/or infrared rays to kill living organisms by means of the radiation chamber (3); and
a sixth stage of returning the fuel to the tank by means of a high-pressure hose (11) with enough pressure to cause a rotating high-pressure jet (10) installed at the end of said hose (11) to work.
